(19) United States
(12) Patent Application Publication
(10) Pub. No.: US 2009/0311731 A1
(43) Pub. Date: Dec. 17, 2009

(54) Modified Molecule

(75) Inventor: Eng Kiat Lim, York (GB)

Correspondence Address: MORGAN, LEWIS & BOCKIUS LLP (SF), One Market, Spear Street Tower, Suite 2800, San Francisco, CA, 94105, US

(73) Assignee: The University of York, Heslington, York (GB)

(21) Appl. No.: 12/302,798

(22) PCT Filed: May 25, 2007
(86) PCT No.: PCT/GB07/01888

§ 371 Date: Aug. 5, 2009

(30) Foreign Application Priority Data
May 26, 2006 (GB) 010498.8

(51) Int. Cl. C12Q 1/48; C12N 1/21; C12M 3/00; C12P 19/02

(52) U.S. Cl. 435/15; 435/252.3; 435/289.1; 435/105

(57) ABSTRACT

We describe a microbial cell, typically a bacterial cell, genetically engineered to produce a modified sugar nucleotide, for example UDP glucuronic acid, and the use of the modified sugar to transfer glucuronic acid to small acceptor molecules.

The invention relates to a microbial cell, typically a bacterial cell, genetically engineered to produce a modified sugar nucleotide, for example UDP glucuronic acid, and the use of the modified sugar to transfer glucuronic acid to small acceptor molecules.

Glycosyltransferases (GTases) are enzymes that post-translationally transfer glycosyl residues from an activated nucleotide sugar to monomeric and polymeric acceptor molecules such as other sugars, proteins, lipids and other organic substrates. These glycosylated molecules take part in diverse metabolic pathways and processes. The transfer of a glycosyl moiety can alter the acceptor's bioactivity, solubility and transport properties within the cell and throughout the organism. The most common activated nucleotide sugar is UDP-glucose which is used by a large number of glucosyltransferase enzymes. Examples of other GTases include rhamnosyltransferases, fucosyltransferases, sialyltransferases, galatosyltransferases and glucuronosyltransferases, each of which use a different donating nucleotide sugar.

Glucuronosyltransferases catalyse the transfer of glucuronic acid from UDP glucuronic acid to small molecule acceptors in both mammalian and plant systems. Typically the small molecules are drugs, environmental chemicals and endogenous substances. In humans the glucuronosyltransferase are grouped into two families; UGT1 and UGT2. UGT1 family members are typically involved in the modification of phenol substrates and bilirubin and some members can modify oestrogens. UGT2 enzymes are subdivided into UGT2A and UGT2B. UGT2A includes genes that encode glucuronosyltransferases expressed by the olfactory epithelium and UGT2B includes genes that encode glucuronosyltransferases that modify bile acids, C19 steroids, C18 steroids, fatty acids, carboxylic acids, phenols and carcinogens. Several members of the UGT2B sub-family have been isolated, for example U.S. Pat. No. 6,287,834 discloses a human glucuronosyltransferase referred to as the UGT2B17. A further example of the isolation and characterisation of glucuronosyltransferase genes is disclosed in WO2006028985.

Plant glucuronosyltransferases are also known. For example, Woo et al (Plant Cell, vol 11, 2303-2315, 1999) describe a Pisum sativum UDP glucuronosyltransferase that is thought to modify flavonoids. The essential function of this glucuronosyltransferase is illustrated by antisense experiments in Medicargo sativa and Arabidopsis thalina which showed cell cycle effects resulting in delayed root emergence, reduced root growth and increased lateral root development (Woo et al Plant Physiology vol 133, 538-548, 2003).

Bacterial expression systems for the production of small molecules, in particular antioxidants, amino acids and peptides, are well known in the art. Typically, bacterial host cells are transformed with a vector that is provided with expression signals that are operably linked to a nucleic acid molecule that encodes a polypeptide sequence the expression of which is desired. Vectors are also provided with replication origins that facilitate the replication of the vector inside the host bacterium. UDP-glucuronic acid is the main nucleotide sugar used by glucuronosyltransferases in mammalian cells for conjugating and detoxifying xenobiotics and steroids. In plants, this sugar nucleotide acts as a precursor for cell wall synthesis. UDP-glucuronic acid is synthesized from UDP-glucose by the enzyme UDP-glucose dehydrogenase (UGD). We disclose the expression of UGD genes in an E. coli system to increase the level of UDP-glucuronic acid in the transformed bacterial cells. This system can be used for production of UDP-glucuronic acid on its own as well as for production of glucuronides when applied in conjunction with glucuronosyltransferases capable of recognising UDP-glucuronic acid as an activated sugar-donor.

According to an aspect of the invention there is provided a microbial cell wherein said cell is genetically modified which modification is the transformation of said cell with a nucleic acid molecule wherein said nucleic acid molecule encodes a polypeptide with the specific enzyme activity associated with a UDP-glucose dehydrogenase.

In a preferred embodiment of the invention said enzyme activity is over-expressed when compared to a non-transformed reference cell of the same species.

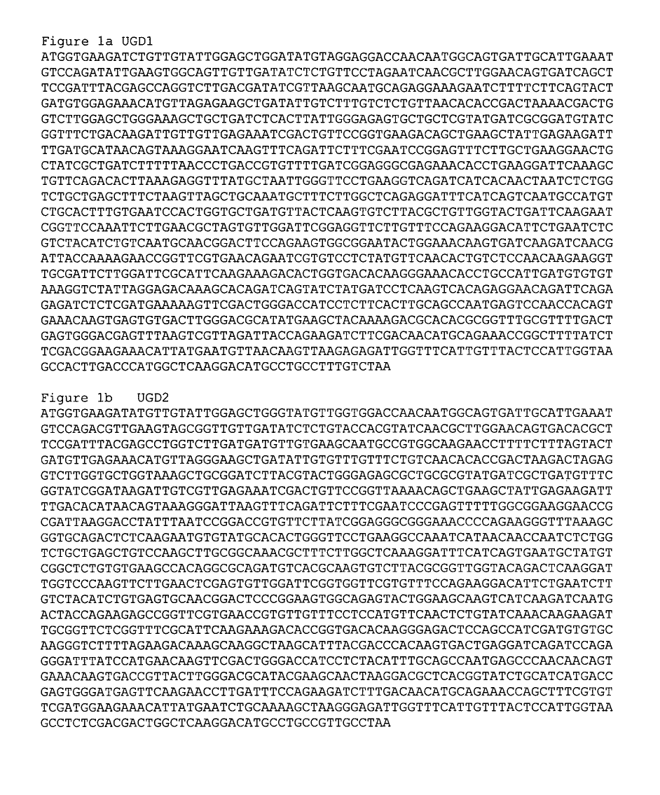

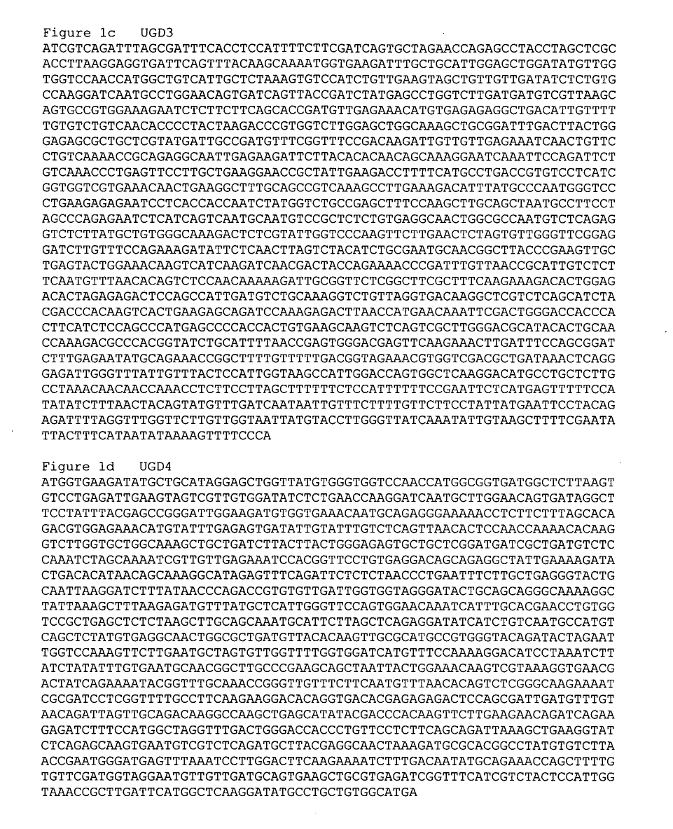

In a preferred embodiment of the invention said nucleic acid molecule is selected from the group consisting of:
i) a nucleic acid molecule comprising a nucleic acid sequence as represented by FIG. 1a, 1b, 1c or 1d;
ii) a nucleic acid molecule which hybridises to the nucleic acid molecule in (i) and which has the enzyme activity associated with UDP-glucose dehydrogenase.

In a further preferred embodiment of the invention said nucleic acid molecule hybridises under stringent hybridisation conditions to the sequence presented in FIG. 1a, 1b, 1c or 1d.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbour Laboratory Press, Cold Spring Harbor, N.Y., 2001); and Tijssen, Laboratory Techniques in Biochemistry and Molecular Biology-Hybridization with Nucleic Acid Probes Part I, Chapter 2 (Elsevier, N.Y., 1993). The Tₘ is the temperature at which 50% of a given strand of a nucleic acid molecule is hybridized to its complementary strand. The following is an exemplary set of hybridization conditions and is not limiting:

Very High Stringency (allows sequences that share
at least 90% identity to hybridize)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency (allows sequences that share
at least 80% identity to hybridize)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency (allows sequences that share
at least 50% identity to hybridize)

| | |
|---|---|
| Hybridization: | 6x SSC at RT to 55° C. for 16-20 hours |
| Wash at least twice: | 2x-3x SSC at RT to 55° C. for 20-30 minutes each. |

In a preferred embodiment of the invention said nucleic acid molecule consists of the nucleic acid sequence presented in FIG. 1a, 1b, 1c or 1d.

In a further preferred embodiment of the invention said cell over-expresses said enzyme activity by at least two-fold when compared to a non-transformed reference cell of the same species. Preferably said enzyme activity is over-expressed at least 3-fold; 4-fold; 5-fold; 6-fold; 7-fold; 8-fold; 9-fold; or at least 10-fold. More preferably said enzyme activity is over-expressed at least 20-fold; 30-fold; 40-fold; or at least 50-fold. Preferably said enzyme activity is over-expressed by at least 100-fold.

The over-expression of enzyme activity can be achieved by means known to those skilled in the art. This could be achieved by placing the gene encoding an enzyme with the activity of UDP-glucose dehydrogenase on a high copy number plasmid. Alternatively, or in addition, said gene can be operably linked to a promoter sequence which provides for high level expression of said gene, said promoter can be constitutively active or inducible. Adaptations also include the provision of selectable markers, which select for cells containing high copy plasmids. These adaptations are well known in the art. There is a significant amount of published literature with respect to expression vector construction and recombinant DNA techniques in general.

In an alternative preferred embodiment of the invention said enzyme over-expression is provided by a variant gene which has the activity of UDP-glucose dehydrogenase wherein said activity is enhanced when compared to an unmodified reference gene as represented by the amino acid sequence in FIG. 1a, 1b, 1c or 1d.

In a preferred embodiment of the invention said cell is transformed with a gene which encodes a variant polypeptide which is modified by addition, deletion or substitution of at least one amino acid residue and wherein said variant polypeptide has the activity associated with UDP-glucose dehydrogenase; preferably said activity is enhanced.

A variant polypeptide may differ in amino acid sequence by one or more substitutions, additions, deletions, truncations that may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine and lysine; e) isoleucine, leucine, methionine and valine and f) phenylalanine, tyrosine and tryptophan. Most highly preferred are variants that retain or enhance the same biological function and activity as the reference polypeptide from which it varies. A functionally equivalent polypeptide is a variant wherein one in which one or more amino acid residues are substituted with conserved or non-conserved amino acid residues, or one in which one or more amino acid residues includes a substituent group. Conservative substitutions are the replacements, one for another, among the aliphatic amino acids Ala, Val, Leu and Ile; interchange of the hydroxyl residues Ser and Thr; exchange of the acidic residues Asp and Glu; substitution between amide residues Asn and Gln; exchange of the basic residues Lys and Arg; and replacements among aromatic residues Phe and Tyr.

In addition, the invention features polypeptide sequences having at least 75% identity with the polypeptide sequences as herein disclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequences illustrated herein.

In a preferred embodiment of the invention said microbial cell is further transformed with a nucleic acid molecule that encodes a glucuronosyltransferase.

In a preferred embodiment of the invention said glucuronosyltransferase is a human glucuronosyltransferase.

In a preferred embodiment of the invention said human glucuronosyltransferase is a UGT 1 glucuronosyltransferase.

In an alternative preferred embodiment of the invention said human glucuronosyltransferase is a UGT2 glucuronosyltransferase.

Preferably, said UGT2 glucuronosyltransferase is a UGT2A or a UGT2B glucuronosyltransferase.

In an alternative preferred embodiment of the invention said glucuronosyltransferase is a plant glucuronosyltransferase.

In a preferred embodiment of the invention said plant glucuronosyltransferase is isolated and selected from the following group of plant species: Artemisia annua, corn (Zea mays), canola (Brassica napus, Brassica rapa ssp.), alfalfa (Medicago sativa), rice (Oryza sativa), rye (Secale cerale), sorghum (Sorghum bicolor, Sorghum vulgare), sunflower (helianthus annuas), wheat (Tritium aestivum), soybean (Glycine max), tobacco (Nicotiana tabacum), potato (Solanum tuberosum), peanuts (Arachis hypogaea), cotton (Gossypium hirsutum), sweet potato (Iopmoea batatus), cassaya (Manihot esculenta), coffee (Cofea spp.), coconut (Cocos nucifera), pineapple (Anana comosus), citris tree (Citrus spp.) cocoa (Theobroma cacao), tea (Camellia senensis), banana (Musa spp.), avacado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), oats, barley, vegetables and ornamentals.

Preferably, plant cells of the present invention are crop plant cells (for example, cereals and pulses, maize, wheat, potatoes, tapioca, rice, sorghum, millet, cassaya, barley, pea, and other root, tuber or seed crops. Important seed crops are oil-seed rape, sugar beet, maize, sunflower, soybean, and sorghum. Horticultural plants to which the present invention may be applied may include lettuce, endive, and vegetable brassicas including cabbage, broccoli, and cauliflower, and carnations and geraniums. The present invention may be applied in tobacco, cucurbits, carrot, strawberry, sunflower, tomato, pepper, chrysanthemum.

Grain plants that provide seeds of interest include oil-seed plants and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava been, lentils, chick pea.

In a preferred embodiment of the invention said plant glucuronosyltransferase is isolated from Medicargo spp.

In a preferred embodiment of the invention said microbial cell is a bacterial cell.

In a further preferred embodiment of the invention said bacterial cell is a Gram negative bacterial cell, for example Escherichia coli.

In an alternative preferred embodiment of the invention said bacterial cell is a Gram positive bacterial cell, for example, a bacterium of the genus Bacillus spp. (e.g. B. subtilis; B. licheniformis; B. amyloliquefaciens).

Gram positive and Gram negative bacteria differ in many respects from one another. A difference exists in the nature of their respective cell walls. The biochemical composition of the B. subtilis cell wall is quite different from that of E. coli. The cell walls of E. coli and B. subtilis contain a framework that is composed of peptidoglycan, a complex of polysaccharide chains covalently cross-linked by peptide chains. This forms a semi-rigid structure that confers physical protection to the cell since the bacteria have a high internal osmotic pressure and can be exposed to variations in external osmolarity. In Gram-positive bacteria, such as the members of the genus Bacillus, the peptidoglycan framework may represent as little as 50% of the cell wall complex and these bacteria are characterised by having a cell wall that is rich in accessory polymers such as teichoic acids. Methods to transform bacteria are well known in the art and have been established for many years. These include chemical methods (e.g. calcium permeabilisation) or physical permeabilisation (e.g. electroporation).

According to a further aspect of the invention there is provided a cell culture vessel comprising a cell according to the invention and media sufficient to support the growth of said cell.

In a preferred embodiment of the invention said vessel is a fermentor.

According to a further aspect of the invention there is provided a method for the manufacture of at least one molecule comprising the steps:
i) providing a vessel comprising a cell according to the invention;
ii) providing cell culture conditions which facilitate the growth of a cell culture contained in said vessel; and optionally
iii) isolating said molecule from said cell or the surrounding growth medium.

In a preferred method of the invention said molecule is UDP glucuronic acid.

In a further preferred method of the invention said vessel includes an aglycone the modification of which by glucuronic acid is desired.

If bacteria are used in the process according to the invention, they are grown or cultured in the manner with which the skilled worker is familiar, depending on the host organism. As a rule, bacteria are grown in a liquid medium comprising a carbon source, usually in the form of sugars, a nitrogen source, usually in the form of organic nitrogen sources such as yeast extract or salts such as ammonium sulfate, trace elements such as salts of iron, manganese and magnesium and, if appropriate, vitamins, at temperatures of between 0° C. and 100° C., preferably between 10° C. and 60° C., while gassing in oxygen.

The pH of the liquid medium can either be kept constant, that is to say regulated during the culturing period, or not. The cultures can be grown batchwise, semi-batchwise or continuously. Nutrients can be provided at the beginning of the fermentation or fed in semi-continuously or continuously. The products produced can be isolated from the bacteria as described above by processes known to the skilled worker, for example by extraction, distillation, crystallization, if appropriate precipitation with salt, and/or chromatography. To this end, the organisms can advantageously be disrupted beforehand.

In this process, the pH value is advantageously kept between pH 4 and 12, preferably between pH 6 and 9, especially preferably between pH 7 and 8.

The culture medium to be used must suitably meet the requirements of the strains in question. Descriptions of culture media for various microorganisms can be found in the textbook “Manual of Methods for General Bacteriology” of the American Society for Bacteriology (Washington D.C., USA, 1981).

As described above, these media which can be employed in accordance with the invention usually comprise one or more carbon sources, nitrogen sources, inorganic salts, vitamins and/or trace elements.

Preferred carbon sources are sugars, such as mono-, di- or polysaccharides. Examples of carbon sources are glucose, fructose, mannose, galactose, ribose, sorbose, ribulose, lactose, maltose, sucrose, raffinose, starch or cellulose. Sugars can also be added to the media via complex compounds such as molasses or other by-products from sugar refining. The addition of mixtures of a variety of carbon sources may also be advantageous. Other possible carbon sources are oils and fats such as, for example, soya oil, sunflower oil, peanut oil and/or coconut fat, fatty acids such as, for example, palmitic acid, stearic acid and/or linoleic acid, alcohols and/or polyalcohols such as, for example, glycerol, methanol and/or ethanol, and/or organic acids such as, for example, acetic acid and/or lactic acid.

Nitrogen sources are usually organic or inorganic nitrogen compounds or materials comprising these compounds. Examples of nitrogen sources comprise ammonia in liquid or gaseous form or ammonium salts such as ammonium sulfate, ammonium chloride, ammonium phosphate, ammonium carbonate or ammonium nitrate, nitrates, urea, amino acids or complex nitrogen sources such as cornsteep liquor, soya meal, soya protein, yeast extract, meat extract and others. The nitrogen sources can be used individually or as a mixture.

Inorganic salt compounds which may be present in the media comprise the chloride, phosphorus and sulfate salts of calcium, magnesium, sodium, cobalt, molybdenum, potassium, manganese, zinc, copper and iron.

Inorganic sulfur-containing compounds such as, for example, sulfates, sulfites, dithionites, tetrathionates, thiosulfates, sulfides, or else organic sulfur compounds such as mercaptans and thiols may be used as sources of sulfur for the production of sulfur-containing fine chemicals, in particular of methionine.

Phosphoric acid, potassium dihydrogenphosphate or dipotassium hydrogenphosphate or the corresponding sodium-containing salts may be used as sources of phosphorus.

Chelating agents may be added to the medium in order to keep the metal ions in solution. Particularly suitable chelating agents comprise dihydroxyphenols such as catechol or protocatechuate and organic acids such as citric acid.

The fermentation media used according to the invention for culturing microorganisms usually also comprise other growth factors such as vitamins or growth promoters, which include, for example, biotin, riboflavin, thiamine, folic acid, nicotinic acid, panthothenate and pyridoxine. Growth factors and salts are frequently derived from complex media components such as yeast extract, molasses, cornsteep liquor and the like. It is moreover possible to add suitable precursors to the culture medium. The exact composition of the media compounds heavily depends on the particular experiment and is decided upon individually for each specific case. Information on the optimization of media can be found in the textbook “Applied Microbiol. Physiology, A Practical Approach” (Editors P. M. Rhodes, P. F. Stanbury, IRL Press (1997) pp. 53-73, ISBN 0 19 963577 3). Growth media can also be obtained from commercial suppliers, for example Standard 1 (Merck) or BHI (brain heart infusion, DIFCO) and the like.

All media components are sterilized, either by heat (20 min at 1.5 bar and 121° C.) or by filter sterilization. The components may be sterilized either together or, if required, separately. All media components may be present at the start of the cultivation or added continuously or batchwise, as desired.

The culture temperature is normally between 15° C. and 45° C., preferably at from 25° C. to 40° C., and may be kept constant or may be altered during the experiment. The pH of the medium should be in the range from 5 to 8.5, preferably around 7.0. The pH for cultivation can be controlled during cultivation by adding basic compounds such as sodium hydroxide, potassium hydroxide, ammonia and aqueous ammonia or acidic compounds such as phosphoric acid or sulfuric acid. Foaming can be controlled by employing antifoams such as, for example, fatty acid polyglycol esters. To maintain the stability of plasmids it is possible to add to the medium suitable substances having a selective effect, for example antibiotics. Aerobic conditions are maintained by introducing oxygen or oxygen-containing gas mixtures such as, for example, ambient air into the culture. The fermentation broths obtained in this way, in particular those comprising polyunsaturated fatty acids, usually contain a dry mass of from 7.5 to 25% by weight.

The fermentation broth can then be processed further. The biomass may, according to requirement, be removed completely or partially from the fermentation broth by separation methods such as, for example, centrifugation, filtration, decanting or a combination of these methods or be left completely in said broth. It is advantageous to process the biomass after its separation.

However, the fermentation broth can also be thickened or concentrated without separating the cells, using known methods such as, for example, with the aid of a rotary evaporator, thin-film evaporator, falling-film evaporator, by reverse osmosis or by nanofiltration. Finally, this concentrated fermentation broth can be processed to obtain the fatty acids present therein.

According to a further aspect of the invention there is provided a screening method to assay the activity of at least one glycosyltransferase polypeptide for glucuronosyltransferase activity with respect to an aglycone comprising the steps of:
i) providing a cell culture vessel comprising a microbial cell according to the invention wherein the vessel includes cell culture media and an aglycone to be tested; and
ii) detecting the presence of a glucuronic acid modified glucoside in said cell culture medium.

According to a further aspect of the invention there is provided a screening method to assay the activity of at least one agent for glucuronosyltransferase modulating activity comprising the steps of:
i) providing a cell culture vessel comprising a microbial cell according to the invention wherein the vessel includes cell culture media, an aglycone and an agent to be tested for glucuronosyltransferase modulating activity; and
ii) detecting the effect, or not, of said agent on the activity of said glucuronosyltransferase.

In a preferred method of the invention said agent is an antagonist of said glucuronosyltransferase.

Antagonistic agents are agents that, either directly or indirectly, inhibit the activity of a glucuronosyltransferase.

In a further preferred method of the invention said method comprises a plurality of glycosyltransferases or glucuronosyltransferase.

Test formats that allow the simultaneous or near simultaneous assaying of a plurality of glycosyltransferases or glucuronosyltransferase are known in the art and include the use of multiwell plates comprising assay reactants. Systems are available for the collation of signals from multiple assays.

In a preferred method of the invention said method further comprises the steps of:
i) collating the data generated in the screen;
ii) converting the collated data into a data analysable form; and optionally
iii) providing an output for the analysed data.

The screening of large numbers of substrates and/or agents requires preparing arrays of cells for the handling and the administration of substrates/agents. Standard multiwell micro titre plates with formats such as 6, 12, 48, 96 and 384 wells are typically used for compatibility with automated loading and robotic handling systems. Typically, high throughput screens use homogeneous mixtures of agents with an indicator compound that is either converted or modified resulting in the production of a signal. The signal is measured by suitable means (for example detection of fluorescence emission, optical density, or radioactivity) followed by integration of the signals from each well containing the cells, substrate/agent and indicator compound. The present invention utilises the detection of a sugar in cell culture medium and this detection may be the result of the direct detection of the sugar or an indirect measure of the concentration of cleaved sugar from a modified substrate.

In a preferred method of the invention said glycosyltransferase is a known glucuronosyltransferase; preferably a human glucuronosyltransferase or a plant glucuronosyltransferase.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

FIG. 1a UDP-glucose dehydrogenase UGD 1; FIG. 1b UDP-glucose dehydrogenase UGD 2; FIG. 1c UDP-glucose dehydrogenase UGD 3; and FIG. 1d UDP-glucose dehydrogenase UGD 4; and

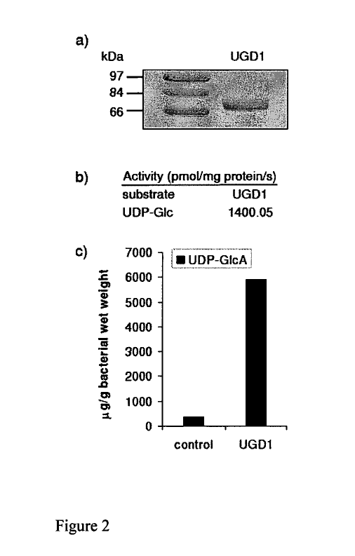

FIG. 2 Analysis of UGD1. (a) SDS-PAGE analysis of recombinant UGD1. (b) The in vitro activity of recombinant UGD1 towards UDP-glucose (Glc). The product UDP-glucuronic acid (GlcA) was confirmed and quantified through ion-pair HPLC-MS analysis. (c) The UDP-GlcA level in the E. coli cells expressing UGD1 protein. Bacterial cells transformed with empty vector were used as a control.

### MATERIALS AND METHODS

### Plasmid Construction

The cDNAs of UGD1 was amplified from an arabidopsis root cDNA library by PCR using the primers listed in Table 1. The cDNAs were cloned into pGEX-2T vector (Amersham Pharmacia) using the BamHI and EcoRI sites. The resulting plasmids allow the UGD1 protein to be expressed as recombinant proteins with a glutathione-S-transferase (GST) fusion at the N-terminus.

Recombinant Protein Purification and Activity Assay.

The plasmids expressing GST-UGD1 fusion proteins were transformed into E. coli BL21 cells for recombinant protein preparation. The cells were grown at 20° C. in 75 ml 2×YT medium containing 50 μg/ml ampicillin to an OD₆₀₀ of 0.8-1.0. The culture was then incubated with 1 mM isopropyl-1-thio-β-D-galactopyranoside (IPTG) for 24 h at 20° C. The cells were harvested (5000 g for 5 min), osmotically shocked, centrifuged again (40,000 g for 30 min). The supernatant was mixed with 100 μl of 50% glutathione-coupled Sepharose (Pharmacia) at room temperature for 30 min. The beads were then washed with phosphate-buffered saline and adsorbed proteins were eluted with 20 mM reduced-form glutathione, 100 mM Tris-HCl (pH 8.0), and 120 mM NaCl, according to the manufacturer's instructions.

The activity of UGD1 recombinant protein was assayed following the methods described by Hinterberg et al¹ with modification. Each reaction mix (200 μl) contained 10 mM Tris-HCl (pH 8.0), 1 mM EDTA (pH 8.0), 1 mM NAD⁺, 1 mM UDP-glucose and 5 μg recombinant proteins. The reaction was carried out at 30° C. for 2 h. The reaction mix was stored at −20° C. before ion-pair HPLC-MS analysis.

Extraction of Nucleotide-Sugars from E. coli

E. coli cell cultures (1 L) expressing UGD1 proteins were harvested and disrupted by French Press (ThermoElectron) in PBS buffer (10 ml). GDP-Glc, which was not detected in the cell lysate, was added to the lysate as the reference for normalization of the nucleotide sugar levels. After centrifugation (40,000×g, 5 min), the supernatant was collected, filtrated through Biomax-30K and Biomax-10K (Millipore), and freeze-dried. The sample was dissolved in H₂O and was analysed by ion-pair HPLC-MS.

Ion-Pair HPLC-MS Analysis

Samples containing nucleotide-sugars were analysed using ion-pair HPLC-MS with a Columbus 5μ C₁₈ column (150×3.2 mm, Phenomenex) at a flow rate of 0.5 ml/min with isocratic 20 mM triethylammoniumacetate (TEAA) buffer (pH 7.0) for 15 min, followed by a linear gradient of 0-2% acetonitrile in 20 mM TEAA buffer over 20 min. The column was then washed with 4% acetonitrile in 20 mM TEAA buffer for 5 min and equilibrated with 20 mM TEAA buffer for 5 min. The chromatography was monitored at 260 nm. Negative ion electrospray MS data were acquired on an Applied Biosystems QSTAR Pulsar i hybrid quadrupole time-of-flight instrument with TurboIonSpray source, scanning the ranges m/z 250-650. Nitrogen was used as nebulisation gas (3.3 L/min). The drying gas flow rate was 6.0 L/min and the temperature was set at 300° C. The ion spray voltage was −2500 V. The data were collected and processed using Analyst QS (Applied Biosystems) software. UDP-glucuronic acid was quantified using the extinction coefficient of authentic UDP-glucuronic acid purchased from Sigma-Aldrich.

### REFERENCES

1. Hinterberg, B., Klos, C., Tenhaken, R. (2002) Plant Physiol. Biochem. 40, 1011-1017.
2. Turner, W., Botha, F. C. (2002) Arch. Biochem. Biophy. 407, 209-216.
3. Seitz, B., Klos, C., Wurm, M., Tenhaken, R. (2000) Plant J. 21, 537-546.
4. Seifert, G. J. (2004) Curr. Opin. Plant Biol. 7, 277-284.

TABLE 1
Summary of primers designed for plasmid
construction

| Primer | Restriction | |
|---|---|---|
| name | site | Sequence (5′-3′) |
| UGD1 | BamHI | CGGGATCCATGGTGAAGATCTGTTGTATTGG |
| forward | | A |
| UGD 1 | EcoRI | CGGAATTCTTAGACAAAGGCAGGCATGTCCT |
| reverse | | T |

Restriction sites within the primers are underlined for clarity.

1. A microbial cell wherein said cell is genetically modified which modification is the transformation of said cell with a nucleic acid molecule wherein said nucleic acid molecule encodes a polypeptide with the specific enzyme activity associated with a UDP-glucose dehydrogenase.

2. A cell according to claim 1 wherein said enzyme activity is over-expressed when compared to a non-transformed cell of the same species.

3. A cell according to claim 1 wherein said nucleic acid molecule is selected from the group consisting of:
i) a nucleic acid molecule comprising a nucleic acid sequence selected from SEQ ID NO: 1-4;
ii) a nucleic acid molecule which hybridises to the nucleic acid molecule in (i) and which has the enzyme activity associated with UDP-glucose dehydrogenase.

4. A cell according to claim 3 wherein said nucleic acid molecule hybridises under stringent hybridisation conditions to the sequence selected from SEQ ID NO: 1-4.

5. A cell according to claim 3 wherein said nucleic acid molecule consists of the nucleic acid sequence selected from SEQ ID NO: 1-4.

6. A cell according to claim 1 wherein said cell over-expresses said enzyme activity by at least two-fold when compared to a non-transformed cell of the same species.

7. A cell according to claim 6 wherein said enzyme over-expression is provided by a variant gene which has the activity of UDP-glucose dehydrogenase wherein said activity is enhanced when compared to an unmodified gene as represented by the sequence selected from SEQ ID NO: 1-4.

8. A cell according to claim 6 wherein said cell is transformed with a gene which encodes a variant polypeptide which is modified by addition, deletion or substitution of at least one amino acid residue and wherein said variant polypeptide has the activity associated with UDP-glucose dehydrogenase.

9. A cell according to claim 1 wherein said microbial cell is further transformed with a nucleic acid molecule that encodes a glucuronosyltransferase.

10. A cell according to claim 9 wherein said glucuronosyltransferase is a human glucuronosyltransferase.

11. A cell according to claim 10 wherein said human glucuronosyltransferase is a UGT 1 glucuronosyltransferase.

12. A cell according to claim 10 wherein said human glucuronosyltransferase is a UGT2 glucuronosyltransferase.

13. A cell according to claim 12 wherein said UGT2 glucuronosyltransferase is a UGT2A or a UGT2B glucuronosyltransferase.

14. A cell according to claim 9 wherein said glucuronosyltransferase is a plant glucuronosyltransferase.

15. A cell according to claim 1 wherein said microbial cell is a bacterial cell.

16. A cell culture vessel comprising a cell according to claim 1 and media sufficient to support the growth of said cell.

17. A vessel according to claim 16 wherein said vessel is a fermentor.

18. A method for the manufacture of at least one molecule comprising the steps:
i) providing a vessel comprising a cell according to claim 1;
ii) providing cell culture conditions which facilitate the growth of a cell culture contained in said vessel; and optionally
iii) isolating said molecule from said cell or the surrounding growth medium.

19. A method according to claim 18 wherein said molecule is UDP glucuronic acid.

20. A method according to claim 18 wherein said vessel includes an aglycone the modification of which by glucuronic acid is desired.

21. A screening method to assay the activity of at least one glycosyltransferase polypeptide for glucuronosyltransferase activity with respect to an aglycone comprising the steps of:
i) providing a cell culture vessel comprising a microbial cell according to the invention wherein the vessel includes cell culture media and an aglycone to be tested; and
ii) detecting the presence of a glucuronic acid modified glucoside in said cell culture medium.

22. A screening method to assay the activity of at least one agent for glucuronosyltransferase modulating activity comprising the steps of:
i) providing a cell culture vessel comprising a microbial cell according to claim 1 wherein the vessel includes cell culture media, an aglycone and an agent to be tested for glucuronosyltransferase modulating activity; and
ii) detecting the effect, or not, of said agent on the activity of said glucuronosyltransferase.

23. A method according to claim 22 wherein said agent is an antagonist of said glucuronosyltransferase.

24. A method according to claim 21 wherein said method comprises a plurality of glycosyltransferases or glucuronosyltransferase.

25. A method according to claim 24 wherein said method further comprises the steps of:
i) collating the data generated in claim 21(ii);
ii) converting the collated data into a data analysable form; and optionally
iii) providing an output for the analysed data.

26. A method according to claim 21 wherein said glycosyltransferase is a known glucuronosyltransferase.

27. A method according to claim 26 wherein said glucuronosyltransferase is a human glucuronosyltransferase.

28. A method according to claim 26 wherein said glucuronosyltransferase is a plant glucuronosyltransferase.


SEQUENCE LISTING (4 sequences)

SEQ ID NO: 1 - DNA, 1443 bp, Arabidopsis thaliana
atggtgaagatctgttgtattggagctggatatgtaggaggaccaacaatggcagtgattgcattgaaatgtccagatattgaagtggcagttgttgatatctctgttcctagaatcaacgcttggaacagtgatcagcttccgatttacgagccaggtcttgacgatatcgttaagcaatgcagaggaaagaatcttttcttcagtactgatgtggagaaacatgttagagaagctgatattgtctttgtctctgttaacacaccgactaaaacgactggtcttggagctgggaaagctgctgatctcacttattgggagagtgctgctcgtatgatcgcggatgtatcggtttctgacaagattgttgttgagaaatcgactgttccggtgaagacagctgaagctattgagaagattttgatgcataacagtaaaggaatcaagtttcagattctttcgaatccggagtttcttgctgaaggaactgctatcgctgatctttttaaccctgaccgtgttttgatcggagggcgagaaacacctgaaggattcaaagctgttcagacacttaaagaggtttatgctaattgggttcctgaaggtcagatcatcacaactaatctctggtctgctgagctttctaagttagctgcaaatgctttcttggctcagaggatttcatcagtcaatgccatgtctgcactttgtgaatccactggtgctgatgttactcaagtgtcttacgctgttggtactgattcaagaatcggttccaaattcttgaacgctagtgttggattcggaggttcttgtttccagaaggacattctgaatctcgtctacatctgtcaatgcaacggacttccagaagtggcggaatactggaaacaagtgatcaagatcaacgattaccaaaagaaccggttcgtgaacagaatcgtgtcctctatgttcaacactgtctccaacaagaaggttgcgattcttggattcgcattcaagaaagacactggtgacacaagggaaacacctgccattgatgtgtgtaaaggtctattaggagacaaagcacagatcagtatctatgatcctcaagtcacagaggaacagattcagagagatctctcgatgaaaaagttcgactgggaccatcctcttcacttgcagccaatgagtccaaccacagtgaaacaagtgagtgtgacttgggacgcatatgaagctacaaaagacgcacacgcggtttgcgttttgactgagtgggacgagtttaagtcgttagattaccagaagatcttcgacaacatgcagaaaccggcttttatcttcgacggaagaaacattatgaatgttaacaagttaagagagattggtttcattgtttactccattggtaagccacttgacccatggctcaaggacatgcctgcctttgtctaa

SEQ ID NO: 2 - DNA, 1443 bp, Arabidopsis thaliana
atggtgaagatatgttgtattggagctgggtatgttggtggaccaacaatggcagtgattgcattgaaatgtccagacgttgaagtagcggttgttgatatctctgtaccacgtatcaacgcttggaacagtgacacgcttccgatttacgagcctggtcttgatgatgttgtgaagcaatgccgtggcaagaaccttttctttagtactgatgttgagaaacatgttagggaagctgatattgtgtttgtttctgtcaacacaccgactaagactagaggtcttggtgctggtaaagctgcggatcttacgtactgggagagcgctgcgcgtatgatcgctgatgtttcggtatcggataagattgtcgttgagaaatcgactgttccggttaaaacagctgaagctattgagaagattttgacacataacagtaaagggattaagtttcagattctttcgaatcccgagtttttggcggaaggaaccgcgattaaggacctatttaatccggaccgtgttcttatcggagggcgggaaaccccagaagggtttaaagcggtgcagactctcaagaatgtgtatgcacactgggttcctgaaggccaaatcataacaaccaatctctggtctgctgagctgtccaagcttgcggcaaacgctttcttggctcaaaggatttcatcagtgaatgctatgtcggctctgtgtgaagccacaggcgcagatgtcacgcaagtgtcttacgcggttggtacagactcaaggattggtcccaagttcttgaactcgagtgttggattcggtggttcgtgtttccagaaggacattctgaatcttgtctacatctgtgagtgcaacggactcccggaagtggcagagtactggaagcaagtcatcaagatcaatgactaccagaagagccggttcgtgaaccgtgttgtttcctccatgttcaactctgtatcaaacaagaagattgcggttctcggtttcgcattcaagaaagacaccggtgacacaagggagactccagccatcgatgtgtgcaagggtcttttagaagacaaagcaaggctaagcatttacgacccacaagtgactgaggatcagatccagagggatttatccatgaacaagttcgactgggaccatcctctacatttgcagccaatgagcccaacaacagtgaaacaagtgaccgttacttgggacgcatacgaagcaactaaggacgctcacggtatctgcatcatgaccgagtgggatgagttcaagaaccttgatttccagaagatctttgacaacatgcagaaaccagctttcgtgttcgatggaagaaacattatgaatctgcaaaagctaagggagattggtttcattgtttactccattggtaagcctctcgacgactggctcaaggacatgcctgccgttgcctaa

SEQ ID NO: 3 - DNA, 1778 bp, Arabidopsis thaliana
atcgtcagatttagcgatttcacctccattttcttcgatcagtgctagaaccagagcctacctagctcgcaccttaaggaggtgattcagtttacaagcaaaatggtgaagatttgctgcattggagctggatatgttggtggtccaaccatggctgtcattgctctaaagtgtccatctgttgaagtagctgttgttgatatctctgtgccaaggatcaatgcctggaacagtgatcagttaccgatctatgagcctggtcttgatgatgtcgttaagcagtgccgtggaaagaatctcttcttcagcaccgatgttgagaaacatgtgagagaggctgacattgtttttgtgtctgtcaacacccctactaagacccgtggtcttggagctggcaaagctgcggatttgacttactgggagagcgctgctcgtatgattgccgatgtttcggtttccgacaagattgttgttgagaaatcaactgttcctgtcaaaaccgcagaggcaattgagaagattcttacacacaacagcaaaggaatcaaattccagattctgtcaaaccctgagttccttgctgaaggaaccgctattgaagaccttttcatgcctgaccgtgtcctcatcggtggtcgtgaaacaactgaaggctttgcagccgtcaaagccttgaaagacatttatgcccaatgggtccctgaagagagaatcctcaccaccaatctatggtctgccgagctttccaagcttgcagctaatgccttcctagcccagagaatctcatcagtcaatgcaatgtccgctctctgtgaggcaactggcgccaatgtctcagaggtctcttatgctgtgggcaaagactctcgtattggtcccaagttcttgaactctagtgttgggttcggaggatcttgtttccagaaagatattctcaacttagtctacatctgcgaatgcaacggcttacccgaagttgctgagtactggaaacaagtcatcaagatcaacgactaccagaaaacccgatttgttaaccgcattgtctcttcaatgtttaacacagtctccaacaaaaagattgcggttctcggcttcgctttcaagaaagacactggagacactagagagactccagccattgatgtctgcaaaggtctgttaggtgacaaggctcgtctcagcatctacgacccacaagtcactgaagagcagatccaaagagacttaaccatgaacaaattcgactgggaccacccacttcatctccagcccatgagccccaccactgtgaagcaagtctcagtcgcttgggacgcatacactgcaaccaaagacgcccacggtatctgcattttaaccgagtgggacgagttcaagaaacttgatttccagcggatctttgagaatatgcagaaaccggcttttgtttttgacggtagaaacgtggtcgacgctgataaactcagggagattgggtttattgtttactccattggtaagccattggaccagtggctcaaggacatgcctgctcttgcctaaacaacaaccaaacctcttccttagcttttttctccattttttccgaattctcatgagtttttccatatatctttaactacagtatgtttgatcaataattgtttcttttgttcttcctattatgaattcctacagagattttaggtttggttcttgttggtaattatgtaccttgggttatcaaatattgtaagcttttcgaatattactttcataatataaaagttttccca

SEQ ID NO: 4 - DNA, 1446 bp, Arabidopsis thaliana
atggtgaagatatgctgcataggagctggttatgtgggtggtccaaccatggcggtgatggctcttaagtgtcctgagattgaagtagtcgttgtggatatctctgaaccaaggatcaatgcttggaacagtgataggcttcctatttacgagccgggattggaagatgtggtgaaacaatgcagagggaaaaacctcttctttagcacagacgtggagaaacatgtatttgagagtgatattgtatttgtctcagttaacactccaaccaaaacacaaggtcttggtgctggcaaagctgctgatcttacttactgggagagtgctgctcggatgatcgctgatgtctccaaatctagcaaaatcgttgttgagaaatccacggttcctgtgaggacagcagaggctattgaaaagatactgacacataacagcaaaggcatagagtttcagattctctctaaccctgaatttcttgctgagggtactgcaattaaggatctttataacccagaccgtgtgttgattggtggtagggatactgcagcagggcaaaaggctattaaagctttaagagatgtttatgctcattgggttccagtggaacaaatcatttgcacgaacctgtggtccgctgagctctctaagcttgcagcaaatgcattcttagctcagaggatatcatctgtcaatgccatgtcagctctatgtgaggcaactggcgctgatgttacacaagttgcgcatgccgtgggtacagatactagaattggtccaaagttcttgaatgctagtgttggttttggtggatcatgtttccaaaaggacatcctaaatcttatctatatttgtgaatgcaacggcttgcccgaagcagctaattactggaaacaagtcgtaaaggtgaacgactatcagaaaatacggtttgcaaaccgggttgtttcttcaatgtttaacacagtctcgggcaagaaaatcgcgatcctcggttttgccttcaagaaggacacaggtgacacgagagagactccagcgattgatgtttgtaacagattagttgcagacaaggccaagctgagcatatacgacccacaagttcttgaagaacagatcagaagagatctttccatggctaggtttgactgggaccaccctgttcctcttcagcagattaaagctgaaggtatctcagagcaagtgaatgtcgtctcagatgcttacgaggcaactaaagatgcgcacggcctatgtgtcttaaccgaatgggatgagtttaaatccttggacttcaagaaaatctttgacaatatgcagaaaccagcttttgtgttcgatggtaggaatgttgttgatgcagtgaagctgcgtgagatcggtttcatcgtctactccattggtaaaccgcttgattcatggctcaaggatatgcctgctgtggcatga